(19) United States
(12) Patent Application Publication
(10) Pub. No.: US 2017/0074954 A1
(43) Pub. Date: Mar. 16, 2017

(54) MAGNETIC RESONANCE-LOCAL COIL ARRANGEMENT WITH AN OPTIMIZED WORKFLOW

(71) Applicant: Robert Rehner, Neunkirchen am Brand (DE)

(72) Inventor: Robert Rehner, Neunkirchen am Brand (DE)

(21) Appl. No.: 15/260,394

(22) Filed: Sep. 9, 2016

(30) Foreign Application Priority Data
Sep. 10, 2015 (DE) 102015217325.9

(51) Int. Cl. G01R 33/34; G01R 33/341; A61B 5/055

(52) U.S. Cl. CPC G01R 33/34007; A61B 5/0555; G01R 33/341; G01R 33/34084

(57) ABSTRACT

A magnetic resonance-local coil arrangement, a patient positioning device and a magnetic resonance device are provided. A magnetic resonance-local coil arrangement includes an assembly unit and a bracket. The bracket has a fixed end and a loose end. The fixed end of the bracket is arranged on the assembly unit. The assembly unit, with a first extension direction, may be arranged on a patient positioning device of a magnetic resonance device.

### CROSS-REFERENCE TO RELATED APPLICATIONS

The present patent document claims the benefit of DE 102015217325.9, filed on Sep. 10, 2015, that is hereby incorporated by reference in its entirety.

### BACKGROUND

The present embodiments relate to a magnetic resonance-local coil arrangement, a patient positioning device, and a magnetic resonance device.

A local coil may have an antenna with at least one antenna element for transmitting and/or receiving magnetic resonance signals and is an essential component in magnetic resonance imaging for obtaining the highest possible image quality by the highest possible Signal-to-Noise Ratio (SNR). However, the correct positioning of the local coil on an object of investigation, such as a patient, requires a high level of preparation time in a measurement preparation phase. Therefore, local coils are often integrated in a patient positioning device to keep the preparation time to a minimum. However, this is only possible for posterior local coils (e.g., local coils that are located under the object of investigation, such as a spinal coil or the lower part of a head coil).

### SUMMARY AND DESCRIPTION

The scope of the present invention is defined solely by the appended claims and is not affected to any degree by the statements within this summary.

The present embodiments may obviate one or more of the drawbacks or limitations in the related art. For example, a device enabling an improved workflow when using local coils (e.g., anterior local coils) is provided.

A magnetic resonance-local coil arrangement with an assembly unit and a bracket on which an antenna may be arranged is provided. The bracket has a fixed end and a loose end, and the fixed end of the bracket is arranged on the assembly unit. The assembly unit, which has a first extension direction, may be arranged on a patient positioning device of a magnetic resonance device.

The magnetic resonance-local coil arrangement may include at least one antenna for transmitting and/or receiving magnetic resonance signals. The at least one antenna may be arranged on the bracket. The antenna may include one or more antenna elements.

The assembly unit serves to arrange the bracket on a patient positioning device that, for example, is configured for the positioning of a patient during a magnetic resonance investigation. The assembly unit may include at least one assembly element for fastening and/or attaching the assembly unit to the patient positioning device.

The fixed end of the bracket may be firmly attached to the assembly unit, or may be detachable. For example, the bracket may be plugged into the assembly unit. The bracket may be pivotable on the assembly unit (e.g., about the first extension direction of the assembly unit).

Extension of an object (e.g., the assembly unit) may be an expansion and/or length of the object (e.g., the length of a curve running from an initial point of the object to an endpoint of the object). The first extension direction may be a main extension direction of the assembly unit that is determined by the assembly unit having a greatest extension parallel to the main extension direction (e.g., the assembly unit is more decisively extended and/or longer in the direction of the main extension direction than in all the directions perpendicular to the main extension direction). This enables a stable arrangement of the bracket on the assembly unit.

The loose end of the bracket may be located on a side of the bracket opposite the fixed end. The loose end may be free. In addition, the loose end may have at least one attachment element (e.g., a hook) to secure, to fasten, and/or to attach the loose end (e.g., detachably).

The bracket may be flexibly configured perpendicular to the first extension direction of the assembly unit.

As seen in cross-section in relation to the first extension direction, the bracket has a modifiable contour. The bracket may include a plurality of bracket parts that are attached to each other at attachment points. The bracket contour may be altered by sliding in or out (e.g., telescopic sliding in or out) and/or tilting of the plurality of bracket parts (e.g., for adjustment to a patient contour).

The bracket may be rigid or flexible in design. At least one bracket part of the plurality of bracket parts may be bendable or flexible around an axis of curvature parallel to the first extension direction. As a result, the bracket and an antenna may be geometrically adjusted to any patient with ease.

Electrical signals, optical signals, and/or energy may be transmitted via the assembly unit.

The assembly unit and the bracket may include electrical and/or optical interfaces by which electrical signals, optical signals, and/or energy may be transmitted between a magnetic resonance device and an antenna of the magnetic resonance-local coil arrangement. The electrical and/or optical connection may be established, for example, by a plug-in action.

The assembly unit may include a first assembly coupling module, and the bracket may include a second assembly coupling module that is arranged on the fixed end of the bracket. The first assembly coupling module and the second assembly coupling module are configured to form an assembly-side coupling unit. This coupling unit includes at least one mechanical fixing device.

The bracket may also be configured as a wireless coil (e.g., the signals are transmitted via radio). In this case, transmission by plug connectors may be at least partially dispensed with.

An embodiment provides that the magnetic resonance-local coil arrangement includes a park mounting unit (e.g., a dimensionally rigid support that is configured to receive the loose end of the bracket). The loose end, and therefore also the entire magnetic resonance-local coil arrangement, are arranged and/or stored in a defined parked position during a measurement preparation phase.

The parked position makes a highly advantageous workflow possible, as the magnetic resonance-local coil arrangement need no longer be disassembled and/or removed from the patient positioning device. After completion of a measurement, the magnetic resonance-local coil arrangement may be brought into the parked position quickly and easily, an examined patient may get down from the patient positioning device, and the next patient may be positioned on the patient positioning device while the magnetic resonance-local coil arrangement remains in the parked position. The magnetic resonance-local coil arrangement may then be brought into a measurement position again in a few acts.

In addition, the park mounting unit may be configured such that any operator may comfortably (e.g., with only one hand) shift the magnetic resonance-local coil arrangement from the parked position into a measurement position without having to reach over the patient positioning device.

Without the need for a lot of technical effort and with the aid of the park mounting unit, the magnetic resonance-local coil arrangement may be prevented from hindering the preparation of the measurement (e.g., the positioning of an object of investigation) as a result of a possible unfavorable position. The risk of the magnetic resonance-local coil arrangement being damaged may be reduced as a result of the defined position of the magnetic resonance-local coil arrangement.

The park mounting unit has at least one attachment element (e.g., a recess for the acceptance of a hook). Based on the at least one attachment element, the loose end of the bracket may be arranged on the park mounting unit. The at least one attachment element of the park mounting unit is coordinated with the at least one attachment element that is arranged at the loose end of the bracket.

The park mounting unit may be arranged on the assembly unit. The park mounting unit may be engaged in the assembly unit. The park mounting unit may be arranged on a patient positioning device of the magnetic resonance device (e.g., the park mounting unit may be arranged directly on the patient positioning device).

The park mounting unit may be configured such that when positioning any object of investigation on a patient positioning device, the largest possible space is provided and/or made available within which positioning may take place to obtain the maximum level of comfort. Thus, an embodiment provides that the park mounting unit is curved around an axis of curvature that is parallel to the first extension direction of the assembly unit. Seen in cross-section in relation to the first extension direction, the park mounting unit may have a modifiable contour.

The shape of the park mounting unit may be such that the park mounting unit may be inserted into a patient reception area of the magnetic resonance device in an operating mode in which the park mounting unit is arranged on the patient positioning device of the magnetic resonance device.

The park mounting unit may be dimensionally rigid in design and/or has sufficient strength to accept the bracket. The park mounting unit may include MR-compatible (e.g., non-imaging) materials in order to avoid artifacts in the magnetic resonance images to be generated. The color of the park mounting unit may be as inconspicuous as possible (e.g., white) and/or is partially or completely transparent.

A further embodiment of the magnetic resonance-local coil arrangement provides that the magnetic resonance-local coil arrangement (e.g., the bracket) may be extended perpendicularly to the first extension direction of the assembly unit. The bracket may have a different extension perpendicular to the first extension direction of the assembly unit (e.g., adopt different extension states). Depending on the extension state, the length of a curve between the loose end of the bracket and the fixed end of the bracket may assume different values. This provides that even with objects of investigation with a wide variation in size, an optimum arrangement of the magnetic resonance-local coil arrangement (e.g., of any antenna) is possible on the object of investigation. The bracket may be configured so that the bracket may change extension by up to 60 centimeters.

The bracket has two or more extendable bracket parts that are mobile relative to each other, enabling the extension of the bracket to be changed. The two or more parts may be locked independently of each other (e.g., via at least one button and/or at least one catch).

The bracket may also be extended by stretching perpendicular to the first extension direction of the assembly unit (e.g., if the bracket housing includes a flexible material). The magnetic resonance-local coil arrangement may include an antenna of flexible material. With the aid of this flexible material of the antenna, the magnetic resonance-local coil arrangement may be geometrically adjusted. For example, the bracket may be arranged on both sides of the flexible antenna, and the bracket itself may also be rigidly configured.

The magnetic resonance-local coil arrangement may include at least one connecting cable. Information and/or energy may be transmitted by the at least one connecting cable (e.g., between any antenna elements of the magnetic resonance-local coil arrangement and a magnetic resonance device).

The bracket may include a bracket housing within which the at least one connecting cable is laid. As a result, the at least one connecting cable may be protected from possible damage. The connecting cable may be laid in the bracket housing invisible to any patient (e.g., the connecting cable is covered by the bracket housing).

The at least one connecting cable has a cable course that enables various extension states of the bracket perpendicular to the first extension direction of the assembly unit. The at least one connecting cable may be laid in a meander-shaped and/or loop-shaped manner (e.g., the at least one connecting cable has a meander-shaped and/or loop-shaped cable coarse). These cable courses are well suited for enabling the extension of the bracket.

A meander-shaped course may be curved and/or undulating. A loop-shaped course may be provided by the connecting cable running in a first direction in a first section and running in a second direction in a second section, with the first direction and the second direction being opposed (e.g., the connecting cable runs at least partially inversely). For example, the first and second directions are perpendicular to the first extension direction of the assembly unit.

The bracket housing may include a cable course for providing a reproducible cable course in various extension states of the local coils.

The bracket may include at least one reel around which the connecting cable may be wound (e.g. independently). In order to wind up the connecting cable independently, the bracket may include one or more springs that may keep the reel under tension.

In a further embodiment, the magnetic resonance-local coil arrangement includes an operation mounting unit where a connection may be established between the loose end of the bracket and the operation mounting unit. The operation mounting unit is configured to accept the loose end of the bracket. The operation mounting unit may, for example, include a groove and/or a rail in which the loose end of the bracket may engage, enabling mechanical anchorage. Mechanical anchorage may dispense with the need for any strap to be attached.

Electrical signals, optical signals, and/or energy may be transmitted via the operation mounting unit. The operation mounting unit and/or the bracket may include electrical and/or optical interfaces based on which electrical signals, optical signals, and/or energy between a magnetic resonance device and the magnetic resonance-local coil arrangement may be transmitted. The electrical and/or optical connection may be established by a plug-in action.

The operation mounting unit includes a first operating coupling module, and the bracket includes a second operating coupling module that is arranged at the loose end of the bracket. The first operating coupling module and the second operating coupling module are configured to form a coupling unit on the operation mounting side. This coupling unit may include at least one mechanical fixing device.

An embodiment of the magnetic resonance-local coil arrangement provides that the magnetic resonance-local coil arrangement is mobile parallel to the first extension direction of the assembly unit. For example, the magnetic resonance-local coil arrangement may be configured to enable the magnetic resonance-local coil arrangement to be pushed along a groove and/or a rail of the patient positioning device. As a result, various areas of an object of investigation may be investigated.

The magnetic resonance-local coil arrangement may include an adapter cable with a plug arranged at the end of the adapter cable. The plug may be plugged into a socket of the magnetic resonance device. Such an adapter cable enables the use of the magnetic resonance-local coil arrangement with existing magnetic resonance devices.

The magnetic resonance-local coil arrangement may include an adapter housing inside which the adapter cable is at least partially arranged. Thus, the adapter cable may be laid so as to be invisible to any patient.

Analogous to the connection, the at least one adapter cable may also be laid in a meander-shaped and/or loop-shaped manner. The magnetic resonance-local coil arrangement may also include at least one reel around which the adapter cable may be wound.

A patient positioning device with at least one magnetic resonance-local coil arrangement is provided.

The patient positioning device may include two grooves and/or rails that may run parallel to each other and/or in a longitudinal direction in relation to the patient positioning device. The magnetic resonance-local coil arrangement may be pushed along these grooves and/or rails so that, for example, a patient's chest may be examined, and after relocation of the magnetic resonance-local coil arrangement (e.g., the local coil) the patient's leg may be examined.

Existing magnetic resonance devices may be modified by an adapter cable. However, the integration of mobile sockets in the patient positioning device may also be provided.

A plurality of magnetic resonance-local coil arrangements may also be operated simultaneously. For example, one of the plurality of magnetic resonance-local coil arrangements (e.g., the shape of the local coil) may be configured for leg imaging (e.g., angiography).

The first extension direction of the assembly unit may be parallel to the longitudinal direction of the patient positioning device. The longitudinal direction of the patient positioning device may be the direction of the longest extension of the patient positioning device.

The bracket of the at least one magnetic resonance-local coil arrangement (e.g., seen in cross-section in relation to the axis of curvature) has a contour that is pivotable around a swivel axis arranged at the edge of the patient table and parallel to the longitudinal direction of the patient positioning device, and is thus adjustable to any object of investigation. As a result, an antenna of the magnetic resonance-local coil arrangement may also be adjusted to the anatomy of the object of investigation.

The patient positioning device may include a table and a base unit, with the table movably mounted in relation to the base unit.

A magnetic resonance device with at least one magnetic resonance-local coil arrangement according to one or more of the present embodiments is also provided.

The magnetic resonance device may include a cylindrical patient reception area (e.g., a bore). The cylinder axis (e.g., the tunnel axis) is also called the z-axis. The first extension direction of the assembly unit is advantageously parallel to the cylinder axis. The at least one magnetic resonance-local coil arrangement may be mobile parallel to the cylinder axis.

The magnetic resonance device may include a patient positioning device. The longitudinal direction of the patient positioning device may be parallel to the cylinder axis. The at least one magnetic resonance-local coil arrangement may be mobile on the patient positioning device of the magnetic resonance device parallel to the cylinder axis.

Any park mounting unit of the magnetic resonance-local coil arrangement may be geometrically adjusted to the cylindrical patient reception area (e.g., the park mounting unit advantageously runs along the curve of the inner wall of the bore). As a result, the park mounting unit may easily be driven into the bore with a maximum amount of space made available for patient positioning.

The advantages of the patient positioning device according to one or more of the present embodiments and/or the magnetic resonance device according to one or more of the present embodiments essentially correspond to the advantages of the magnetic resonance-local coil arrangement according to one or more of the present embodiments, which are described above in detail. Features, advantages, or alternative embodiments mentioned here may likewise also be transmitted to the other objects, and vice versa.

Further advantages, features and details emerge from the exemplary embodiments described hereinafter and on the basis of the diagrams. Corresponding parts are given the same reference characters in all the figures.

### BRIEF DESCRIPTION OF THE DRAWINGS

FIG. 1 depicts a magnetic resonance device with two magnetic resonance-local coil arrangements.

FIG. 2 depicts a cutaway view of a magnetic resonance-local coil arrangement on a patient table in a parked position.

FIG. 3 depicts a cutaway view of a magnetic resonance-local coil arrangement on a patient table in a first operating position.

FIG. 4 depicts a cutaway view of a magnetic resonance-local coil arrangement on a patient table in a second operating position.

FIG. 5 depicts a top view of a magnetic resonance-local coil arrangement on a patient table.

FIG. 6 depicts a bracket with a connecting cable laid in a meander-shaped manner in a first extension state.

FIG. 7 depicts a bracket with a connecting cable laid in a meander-shaped manner in a second extension state.

FIG. 8 depicts a bracket with a connecting cable laid in a loop-shaped manner in a first extension state.

FIG. 9 depicts a bracket with a connecting cable laid in a loop-shaped manner in a second extension state.

FIG. 10 depicts a bracket with a coiled connecting cable in a first extension state.

FIG. 11 depicts a bracket with a coiled connecting cable in a second extension state.

### DETAILED DESCRIPTION

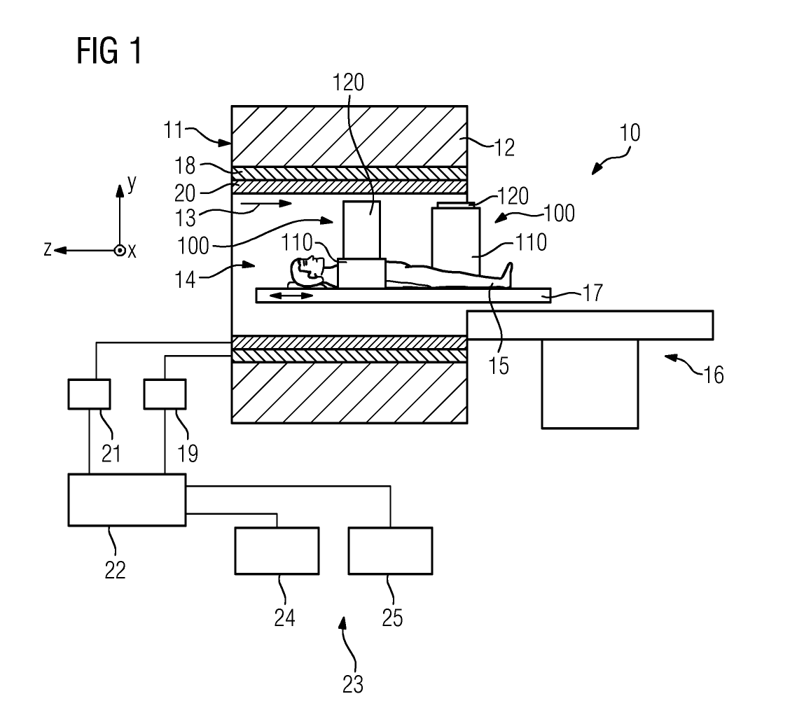

FIG. 1 is a diagrammatic view of a magnetic resonance device 10. The magnetic resonance device 10 includes a magnet unit 11 that has a superconducting main magnet 12 for the generation of a strong and, for example, time-constant main magnetic field 13. In addition, the magnetic resonance device 10 includes a patient reception area 14 for the reception of a patient 15. The patient reception area 14 is cylindrical in design and is cylindrically surrounded by the magnet unit 11 in a peripheral direction. Other embodiments of the patient reception area 14 may be provided. The patient 15 may be pushed into the patient reception area 14 by a patient positioning device 16 of the magnetic resonance device 10. The patient positioning device 16 has a movable patient table 17 inside the patient reception area 14 for this purpose.

The magnet unit 11 has a gradient coil unit 18 for the generation of magnetic field gradients used for spatial encoding during imaging. The gradient coil unit 18 is controlled by a gradient control unit 19 of the magnetic resonance device 10. The magnet unit 11 includes an RF antenna unit 20 that, in this exemplary embodiment, is configured as a body coil permanently integrated in the magnetic resonance device 10. The RF antenna unit 20 is configured for the excitation of atomic nuclei occurring in the main magnetic field 13 generated by the main magnet 12. The RF antenna unit 20 is controlled by an RF antenna control unit 21 of the magnetic resonance device 10 and beams high-frequency magnetic resonance sequences into an investigation area that is essentially formed by a patient reception area 14 of the magnetic resonance device 10. The RF antenna unit 20 is configured to receive magnetic resonance signals.

To control the main magnet 12, the gradient control unit 19, and the RF antenna control unit 21, the magnetic resonance device 10 has a system control unit 22. The system control unit 22 centrally controls the magnetic resonance device 10 (e.g., performing a predetermined imaging gradient-echo sequence). In addition, the system control unit 22 includes an analysis unit (not shown in more detail) for the analysis of medical image data compiled during the magnetic resonance investigation. The magnetic resonance device 10 includes a user interface 23 connected to the system control unit 22. Control information (e.g., imaging parameters) and reconstructed magnetic resonance images may he displayed on a display unit 24 (e.g., on at least one monitor) of the user interface 23 for medical operating personnel. The user interface 23 has an input unit 25 by which information and/or parameters may be entered by medical operating personnel during a measurement procedure.

The magnetic resonance device 10 has two magnetic resonance-local coil arrangements 100 arranged on the patient positioning device 16 (e.g., on the patient table 17). The magnetic resonance-local coil arrangement 100 shown on the left of FIG. 1 is positioned in a chest area of the patient 15 while the magnetic resonance-local coil arrangement 100 shown on the right of FIG. 1 is positioned in a leg area. Each of the magnetic resonance-local coil arrangements 100 includes a bracket 110 for an antenna (not shown in more detail) and a park mounting unit 120. The bracket 110 of the magnetic resonance-local coil arrangement 100 arranged in the chest area is placed around the body of the patient 15 (e.g., this magnetic resonance-local coil arrangement 100 is in an operating state in which efficient magnetic resonance signals may be detected). The bracket 110 of the magnetic resonance-local coil arrangement 100 arranged in the leg area of the patient 15 is in a parked state.

The magnetic resonance device 10 shown in this exemplary embodiment may include further components typically present in magnetic resonance devices. In addition, a general mode of operation of a magnetic resonance device 10 is known to a person skilled in the art.

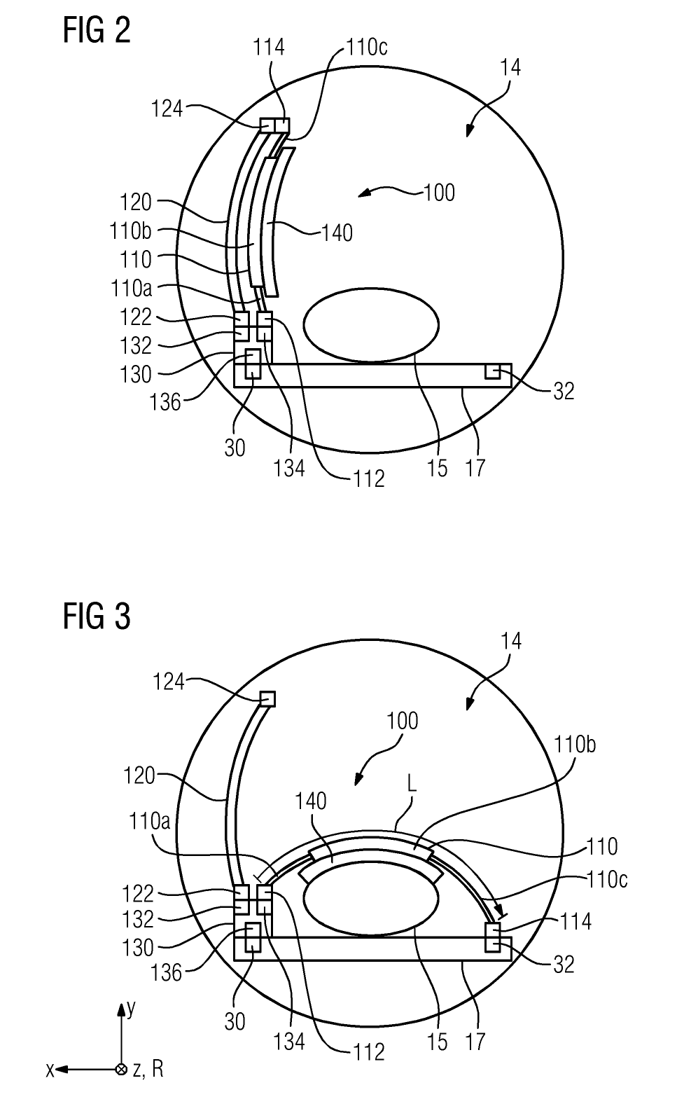

FIGS. 2 to 4 show a diagrammatically detailed cutaway view of a magnetic resonance-local coil arrangement 100 on a patient table 17 in different operating modes. In these figures, the magnetic resonance-local coil arrangement 100 is located inside a cylindrical patient reception area 14 typical of magnetic resonance devices. The magnetic resonance-local coil arrangement 100 includes a bracket 110 on which an antenna 140 is arranged, and an assembly unit 130 that includes a first assembly coupling module 134. In this embodiment, the bracket includes three parts (e.g., 110a, 110b and 110c), an attachment element 114 on a loose end, and a second assembly coupling module 112 on a fixed end. A connection may be established between the fixed end of the bracket 110 and the assembly unit 130 via the first assembly coupling module 134 and the second assembly coupling module 112.

The magnetic resonance-local coil arrangement 100 includes a park mounting unit 120 configured to accept the loose end of the bracket, as shown in FIG. 2. The park mounting unit 120 includes an attachment element 124 that may be detachably connected to the attachment element 114 of the bracket 110.

In this embodiment, the park mounting unit 120 is arranged via an attachment element 122 on the assembly unit 130 that has an attachment element 132 on which the attachment element 122 may be fastened. In one embodiment, the park mounting unit 120 is arranged directly on the patient table 17.

The park mounting unit 120 and/or the bracket 110 may also be directly connected to the assembly unit 130 (e.g., without connecting elements 122 and 132 and/or assembly coupling modules 112 and 134).

The assembly unit 130 includes an assembly element 136. The assembly unit 130 may be fastened to the patient table 17 by a further assembly element 30 permanently integrated in the patient table 17. The assembly unit 130 may also be fastened detachably to this patient table 17.

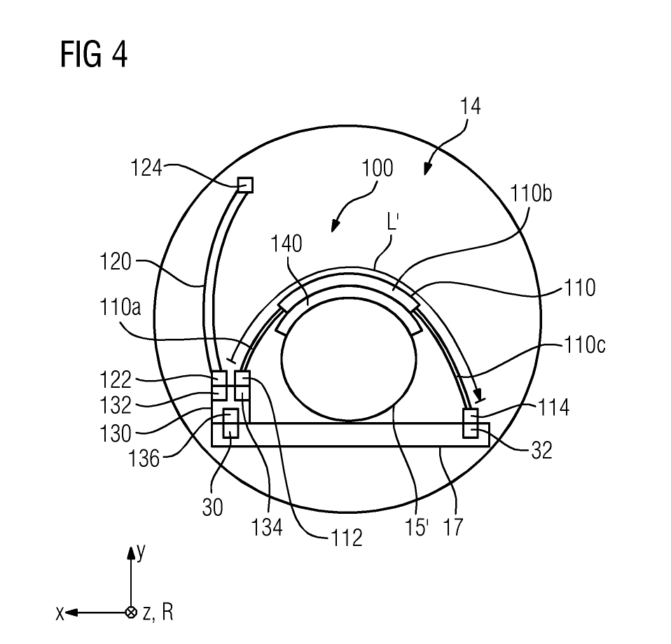

In FIG. 2, the bracket 110, and consequently the magnetic resonance-local coil arrangement, is in a parked position with the loose end of the bracket 110 accepted by the park mounting unit 124. In FIGS. 3 and 4, the bracket 110 is in operating positions with the antenna 140 arranged close to the patient 15, The changeover from the parked position to the operating position may take place by releasing the loose end of the bracket 110 from the park mounting unit 120, pulling the loose end of the bracket 110 over the patient 15, and arranging the loose end of the bracket 110 on an operation mounting unit 32. The operation mounting unit 32 may be permanently integrated in the patient table 17 or may be fastened detachably on the patient table 17.

Electrical signals, optical signals, and/or energy may be transmitted via the operation mounting unit 32 (e.g., to the magnetic resonance device 10). FIG. 3 shows a variant in which electrical signals, optical signals, and/or energy are transmitted via the assembly unit 130. The magnetic resonance-local coil arrangement 100 includes an adapter cable 170 that has a plug 175 at an end that is inserted into a socket 40 of the magnetic resonance device 10. Such customarily existing sockets are mostly arranged at the head end or at the foot end (as depicted) of the patient table 17. In this way, the magnetic resonance-local coil arrangement 100 may easily be made usable for existing magnetic resonance devices 10.

The magnetic resonance-local coil arrangement 100 has an adapter housing 180 inside which the adapter cable 170 is at least partially arranged. As a result, the adapter cable 170 may be laid invisibly.

In FIG. 5, the assembly unit 130 has a first extension direction R. In this embodiment, the assembly unit 130 has a greatest extension parallel to the extension direction R. The extension direction R is parallel to the longitudinal direction of the patient table 17, which is parallel to the cylinder axis z of the reception area 14 (e.g., the bore).

A possible park mounting unit is depicted in FIG. 5.

FIGS. 2 to 4 illustrate that the park mounting unit 120 is curved around an axis of curvature that is parallel to the first extension direction R of the assembly unit 130. The shape of the park mounting unit 120 may be adjusted to the shape of the patient reception area 14 (e.g., to the inner contour of the bore) to enable the introduction of the patient table 17 together with the magnetic resonance-local coil arrangement 100 into the patient reception area 14 and to keep any restrictions to positioning and/or positioning of the patient 15 to a minimum.

FIGS. 2 to 4 show that the bracket 110 is flexibly configured perpendicular to the first extension direction R of the assembly unit 130. As seen in a cross-section to the first extension direction R, the bracket has a modifiable contour. Thus, the contour in the park position in FIG. 2 is different in design to the operating positions in FIGS. 3 and 4. For example, the relative position of the bracket parts 110a, 110b and 110c is variable, resulting in different contours.

FIGS. 2 to 4 show that the bracket 110 may be extended perpendicular to the first extension direction R of the assembly unit 130 (e.g., the extension length of the bracket perpendicular to the first extension direction is variable). The extension length is therefore shorter in the park position than in the operating position in which the loose end of the bracket 110 is connected to the operation mounting unit 32. The extension length may be changed by pushing the local coil parts 110a, 110b and 110c in or out. In one embodiment, flexible materials that permit a change in length may be used for the bracket 110a.

FIGS. 3 and 4 illustrate that the magnetic resonance-local coil arrangement 100 may be flexibly adjusted to a contour of the patient 15. Thus, the patient 15 in FIG. 3 has a greater scope than the patient 15′ in FIG. 4. As a result of the contour-dependent pushing in or out of the bracket parts 110a, 110b and 110c, the extension L and L′ of the bracket changes perpendicular to the first extension direction R of the assembly unit 130. The bracket thus adapts to the contour of the patient 15 to optimize the positioning of the antenna 140 on the patient 15 in both cases.

The magnetic resonance-local coil arrangement 100 includes a connecting cable 150 for transmitting signals and/or energy inside the bracket. Analogous to the adapter housing 180, the bracket 110 may also include a bracket housing inside which the connecting cable 150 is arranged. By this, an outwardly wireless anterior magnetic resonance-local coil arrangement may be realized. In addition, the risk of a cable defect may be reduced as a result of a secure cable course inside housing.

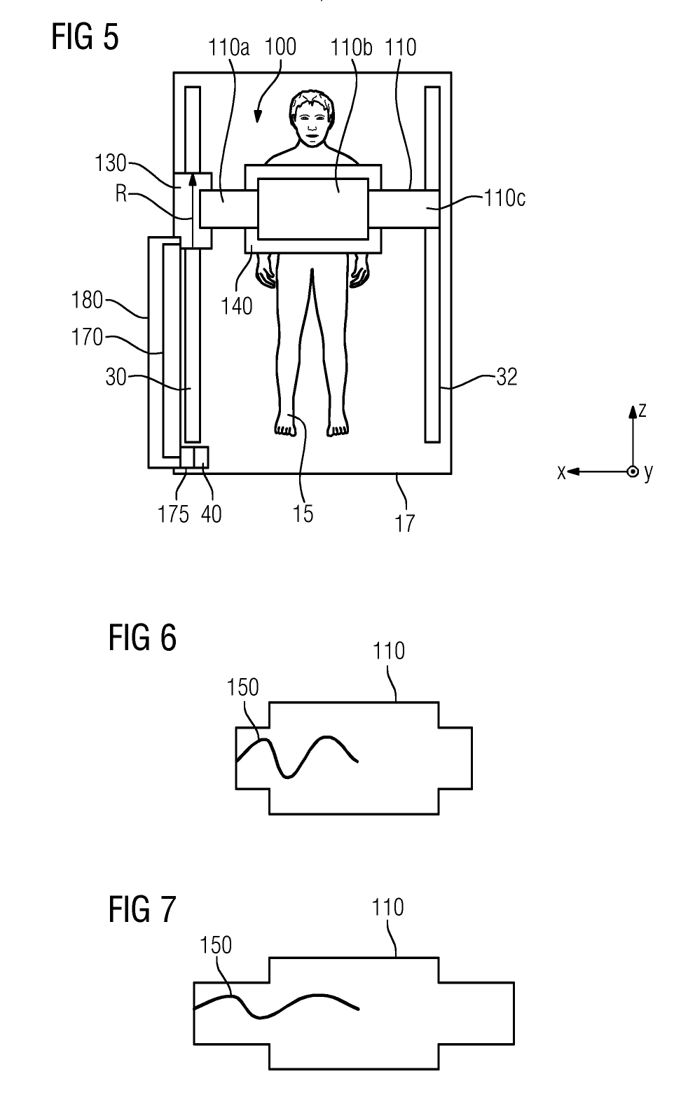

FIGS. 6 to 11 show various options by which the connecting cable 150 may be adjusted to various extension states of the bracket 110. FIGS. 6 and 7 show the connecting cable 150 laid in a meander-shaped manner. The state shown in FIG. 6 may be converted to the state shown in FIG. 7 by removing the bracket 110. A sufficient length of cable is also available in the extended state for meander-shaped routing.

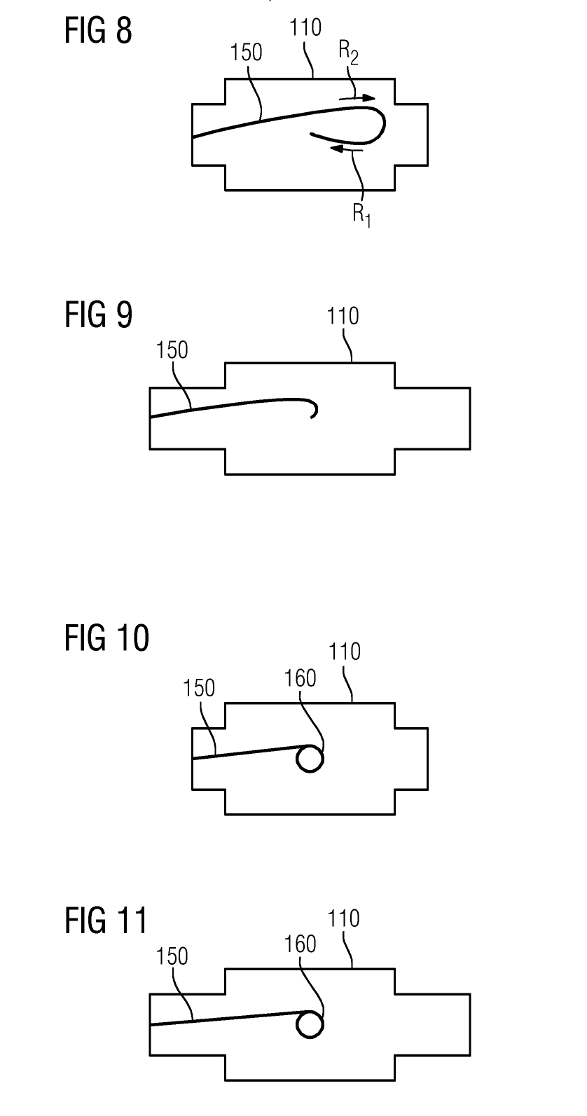

Another option is shown in FIGS. 8 and 9, in which loop-shaped routing is shown (e.g., at least in a contracted state). As illustrated in FIG. 8, the cable runs in sections in different directions R₁ and R₂. Thus, when the connecting cable 150 is laid in a loop-shaped manner in an extended state, a sufficient length of cable is also available, as shown in FIG. 9.

FIGS. 10 and 11 depict the bracket 110 including a reel around which the connecting cable may be wound.

The options for the cable course shown in FIGS. 6 to 11 may also be transmitted to the adapter cable 170 shown in FIG. 5. This may be useful for an embodiment in which the magnetic resonance-local coil arrangement 10 is mobile parallel to the first extension direction R of the assembly unit 130, enabling various areas of the patient's body to be investigated. As a result of such a shift along the z-axis, a transmission path of the adapter-cable 170 is also changed. Thus, for example, as shown in FIG. 5 with the positioning of the magnetic resonance-local coil arrangement 10 on the legs of the patient 15, the transmission path between the assembly unit 130 and the socket 40 at the foot end is smaller than when the magnetic resonance-local coil arrangement 10 is positioned on the chest.

The aforementioned products described in detail are only exemplary embodiments that may be modified in all sorts of ways by a person skilled in the art without departing from the scope of the invention. The use of the indefinite article “a” does not rule out the features concerned also existing repeatedly. The terms “unit” and “module” do not rule out the components concerned consisting of a plurality of interacting subcomponents that may also be spatially distributed if necessary.

The elements and features recited in the appended claims may be combined in different ways to produce new claims that likewise fall within the scope of the present invention. Thus, whereas the dependent claims appended below depend from only a single independent or dependent claim, it is to be understood that these dependent claims may, alternatively, be made to depend in the alternative from any preceding or following claim, whether independent or dependent. Such new combinations are to be understood as forming a part of the present specification.

While the present invention has been described above by reference to various embodiments, it should be understood that many changes and modifications can be made to the described embodiments. It is therefore intended that the foregoing description be regarded as illustrative rather than limiting, and that it be understood that all equivalents and/or combinations of embodiments are intended to be included in this description.

1. A magnetic resonance-local coil arrangement comprising;
an assembly unit; and
a bracket,
wherein the bracket has a fixed end and a loose end,
wherein the fixed end of the bracket is arranged on the assembly unit,
wherein the assembly unit is arrangeable on a patient positioning device of a magnetic resonance device, and
wherein the assembly unit has a first extension direction.

2. The magnetic resonance-local coil arrangement of claim 1, wherein the bracket is flexibly configured to be perpendicular to the first extension direction of the assembly unit.

3. The magnetic resonance-local coil arrangement of claim 1, wherein electrical signals, optical signals, energy, or any combination thereof is transmittable via the assembly unit.

4. The magnetic resonance-local coil arrangement of claim 1, wherein the magnetic resonance-local coil arrangement comprises a park mounting unit configured to accept the loose end of the bracket.

5. The magnetic resonance-local coil arrangement of claim 4, wherein the park mounting unit is curved around an axis of curvature that is parallel to the first extension direction of the assembly unit.

6. The magnetic resonance-local coil arrangement of claim 1, wherein the magnetic resonance-local coil arrangement is extendable perpendicular to the first extension direction of the assembly unit.

7. The magnetic resonance-local coil arrangement of claim 1, wherein the magnetic resonance-local coil arrangement comprises at least one connecting cable.

8. The magnetic resonance-local coil arrangement of claim 7, wherein the bracket comprises a bracket housing, and
wherein the at least one connecting cable is arranged inside the bracket housing.

9. The magnetic resonance-local coil arrangement of claim 7, wherein the at least one connecting cable is laid in a meander-shaped manner, a loop-shaped manner, or the meander-shaped manner and the loop-shaped manner.

10. The magnetic resonance-local coil arrangement of claim 7, wherein the bracket comprises at least one reel around which the connecting cable is windable.

11. The magnetic resonance-local coil arrangement of claim 1, further comprising an operation mounting unit,
wherein a connection is establishable between the loose end of the bracket and the operation mounting unit.

12. The magnetic resonance-local coil arrangement of claim 11, wherein electrical signals, optical signals, energy, or any combination thereof is transmittable via the operation mounting unit.

13. The magnetic resonance-local coil arrangement of claim 1, wherein the magnetic resonance-local coil arrangement is movable in parallel to the first extension direction of the assembly unit.

14. The magnetic resonance-local coil arrangement of claim 1, further comprising an adapter cable,
wherein the adapter cable comprises a plug arranged at an end of the adapter cable, and
wherein the plug is pluggable into a socket of the magnetic resonance device.

15. The magnetic resonance-local coil arrangement of claim 14, further comprising an adapter housing,
wherein the adapter cable is at least partially arranged inside the adapter housing.

16. The magnetic resonance-local coil arrangement of claim 15, wherein the at least one adapter cable is laid in a meander-shaped manner, a loop-shaped manner, or the meander-shaped manner and the loop-shaped manner.

17. The magnetic resonance-local coil arrangement of claim 13, further comprising at least one reel around which the adapter cable is windable.

18. A patient positioning device comprising:
a magnetic resonance-local coil arrangement comprising:
an assembly unit; and
a bracket,
wherein the bracket has a fixed end and a loose end,
wherein the fixed end of the bracket is arranged on the assembly unit,
wherein the assembly unit is arrangeable on a patient positioning device of a magnetic resonance device, and
wherein the assembly unit has a first extension direction.

19. A magnetic resonance device comprising:
a magnetic resonance-local coil arrangement comprising:
an assembly unit; and
a bracket,
wherein the bracket has a fixed end and a loose end,
wherein the fixed end of the bracket is arranged on the assembly unit,
wherein the assembly unit is arrangeable on a patient positioning device of a magnetic resonance device, and
wherein the assembly unit has a first extension direction.
